(19) United States
(12) Patent Application Publication
(10) Pub. No.: US 2024/0368704 A1
(43) Pub. Date: Nov. 7, 2024

(54) METHODS FOR DETECTING CANCER USING CIRCULATING CELL-FREE DNA

(71) Applicant: Zoetis Services LLC, Parsippany, NJ (US)

(72) Inventors: Daniel Grosu, La Jolla, CA (US); Ilya Chorney, La Jolla, CA (US); Wai Yi Tsui, La Jolla, CA (US); Kristina Kruglyak, La Jolla, CA (US); John Tynan, La Jolla, CA (US)

(21) Appl. No.: 18/655,845

(22) Filed: May 6, 2024

Related U.S. Application Data
(60) Provisional application No. 63/500,478, filed on May 5, 2023.

(51) Int. Cl. C12Q 1/6886

(52) U.S. Cl. CPC C12Q 1/6886

(57) ABSTRACT

Provided herein are methods for measuring concentration of circulating cell free DNA (cfDNA) from a sample of a subject, for the purposes of cancer or tumor detection and/or characterization.

### CROSS REFERENCE TO RELATED APPLICATIONS

This application claims priority from U.S. Provisional Application No. 63/500,478 filed May 5, 2023, the contents of which are herein incorporated by reference in its entirety.

### FIELD

The present disclosure relates to methods for detecting or characterizing cancer or a tumor in a subject by optimizing cfDNA concentration thresholds. In particular, the methods provided herein relate to evaluating performance of those thresholds for determining the presence or absence of cancer.

### BACKGROUND

Companion animals, such as dogs and cats are enjoying longer lifespans as veterinary medicine continues to improve. However, this increased lifespan has led to a higher rate of cancers among companion animals. By some estimates, over 50% of dogs over ten years of age are going to die from a cancer-related health issue. Cats are also susceptible to a variety of cancers. Hence, veterinary oncology is required to provide early and effective cancer detection. Among the most common cancers in these animals are lymphoma, hemangiosarcoma (cancer in the blood vessels), histiocytic sarcoma, squamous cell carcinoma (skin cancer), mammary cancer, mast cell tumors, oral tumors, fibrosarcoma (soft tissue cancer), osteosarcoma (bone cancer), pulmonary carcinoma, intestinal adenocarcinoma, pancreatic adenocarcinoma, liver carcinoma, anal sac adenocarcinoma, and urinary tract cancer.

Companion animals, including various breeds of dog, are susceptible to particular cancers (Rafalko, 2023). For example, larger dogs are more susceptible to developing osteosarcoma. German Shepherds, Golden Retrievers, Labrador Retrievers, Pointers, Boxers, English Settlers, Great Danes, Poodles, and Siberian Huskies are susceptible to developing hemangiosarcoma (HSA). HSA tends to affect large breed animals more often than smaller ones. Unfortunately, there are no good tumor biomarkers for cancer, and detection and diagnosis of cancer is often difficult, with invasive surgery and biopsy typically required to make a diagnosis.

Current methods of cancer diagnosis in companion animals include imaging, fine needle aspiration cytology, and biopsies. Liquid biopsies, including blood tests for detecting cancers, offer diagnostic information that is otherwise only accessible through invasive, risky, biopsies. The first applications of liquid biopsies are based on the detection of genetic markers such as sex differences, genetic polymorphisms, or mutations.

Cancer liquid biopsies can also provide information about circulating cell-free DNA (cfDNA), which is extracellular DNA released into the bloodstream predominantly as a result of apoptosis, necrosis, and secretion. Elevated levels of cfDNA have been associated with physiological conditions, including the presence of cancer or tumor. Several studies have demonstrated that increased plasma cfDNA concentrations are associated with dogs with immune-mediated hemolytic anemia, cancer, sepsis, gastric dilation-volvulus syndrome, and trauma. However, compared to literature in human, there is relatively very little literature about the relationship between cfDNA concentrations and cancer in veterinary medicine. The currently available techniques with determining the cfDNA in veterinary medicine are risky and expensive. There is also a paucity of a blood-based noninvasive test that provide a relatively inexpensive and simple way to perform cancer detection accurately.

### SUMMARY OF THE INVENTION

Described herein are methods for measuring and optimizing cfDNA concentration thresholds from a sample from a subject. In some embodiments, the methods are used for improving detection, diagnosis, and screening of cancer in a subject.

Some embodiments provided herein relate to methods detecting a cancer or tumor in a subject. In some embodiments, the methods include isolating circulating cell free DNA (cfDNA) from a sample from the subject, extracting the cfDNA from the sample, determining a concentration of the cfDNA from the sample, generating a first experimental model from one or more cfDNA concentration distributions of subjects with cancer, generating a second experimental model from one or more cfDNA concentration distributions of subjects without cancer, and/or determining the presence of the cancer or tumor based upon the comparison of the first and second models to the concentration of the subjects cfDNA. In some embodiments, the sample is blood, plasma, urine, saliva, effusion, or cerebral spinal fluid. In some embodiments, the determination of the concentration of the cfDNA is done using an electrophoresis solution. In some embodiments, the determination of the concentration of the cfDNA is done using quantitative PCR (qPCR), digital PCR, or fluorometric assays. In some embodiments, one or more optimized threshold is obtained from the first and second experimental model. In some embodiments, the one or more optimized threshold classifies the concentration of the cfDNA into a low, moderate, and high category. In some embodiments, the methods further include performing a genomic cancer screening assay on a subject classified as moderate. In some embodiments, the one or more optimized threshold is different for a given demographic variable. In some embodiments, the demographic variable is subject gender, subject size, subject age, subject breed, and/or spay or neutered subjects. In some embodiments, the one or more optimized threshold is a single threshold. In some embodiments, the concentration of the cfDNA above the threshold is a prediction of cancer. In some embodiments, the subject is a mammal. In some embodiments, the subject is a canine, feline, equine, or human. In some embodiments, the cancer is lymphoma, hemangiosarcoma, soft tissue sarcoma, mast cell tumor, osteosarcoma, mammary gland carcinoma, anal sac adenocarcinoma, and/or malignant melanoma.

Some embodiments provided herein relate to methods of detecting a cancer or tumor in a subject. In some embodiments, the methods include obtaining a biological sample including circulating cell-free DNA (cfDNA) from the subject, determining a concentration of the cfDNA directly from the sample, generating a first experimental model from one or more cfDNA concentration distributions of subjects with cancer, generating a second experimental model from one or more cfDNA concentration distributions of subjects without cancer, and/or determining the presence of the cancer or tumor based upon the comparison of the first and second models to the concentration of the subjects cfDNA. In some embodiments, the sample is blood, plasma, urine, saliva, effusion, or cerebral spinal fluid. In some embodiments, the determination of the concentration of the cfDNA is done using an electrophoresis solution. In some embodiments, the determination of the concentration of the cfDNA is done using quantitative PCR (qPCR), digital PCR, or fluorometric assays. In some embodiments, one or more optimized threshold is obtained from the first and second experimental model. In some embodiments, the one or more optimized threshold classifies the concentration of the cfDNA into a low, moderate, and high category. In some embodiments, the methods further include performing a genomic cancer screening assay on a subject classified as moderate. In some embodiments, the one or more optimized threshold is different for a given demographic variable. In some embodiments, the demographic variable is subject gender, subject size, subject age, subject breed, and/or spay or neutered subjects. In some embodiments, the one or more optimized threshold is a single threshold. In some embodiments, the concentration of the cfDNA above the threshold is a prediction of cancer. In some embodiments, the subject is a mammal. In some embodiments, the subject is a canine, feline, equine, or human. In some embodiments, the cancer is lymphoma, hemangiosarcoma, soft tissue sarcoma, mast cell tumor, osteosarcoma, mammary gland carcinoma, anal sac adenocarcinoma, and/or malignant melanoma.

In some embodiments of the methods described herein, the analysis is pan-cancer.

### BRIEF DESCRIPTION OF THE DRAWINGS

In order to describe the manner in which the above-recited and other advantages and features of embodiments described herein can be obtained, a more particular description will be rendered by reference to specific embodiments thereof which are illustrated in the appended drawings. Understanding that these drawings depict only typical embodiments and are not therefore to be considered to be limiting of its scope, the disclosure will be described and explained with additional specificity and detail through the use of the accompanying drawings in which:

FIGS. 1A-1B shows exemplary flowcharts depicting threshold analysis through a first analysis (FIG. 1A) and a second analysis (FIG. 1B).

FIGS. 2A-2C show exemplary ROC curve demonstrating performance of Concepts 1 and 2 of FIGS. 1A and 1B, as a function of demographic variables, including sex (FIG. 2A), size (FIG. 2B), and age (FIG. 2C). In FIG. 2A, the light line is for the male subjects and the dark line is for the female subjects. In FIG. 2B, the light line is for above the median size of the subjects and the dark line is for below the median size of the subjects. In FIG. 2C, the light line is for above the median age of the subjects and the dark line is for below the median age of the subjects.

FIG. 3 shows an exemplary bar graph of estimated fractions of cancer-diagnosed subjects assigned to each probability tiers, called “Cancer Probability Index” (low, moderate, high) for difficult-to-diagnose (D2D) cancer sets.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein. All references cited herein are expressly incorporated by reference herein in their entirety and for the specific disclosure referenced herein.

Previous studies demonstrated that elevated level of plasma cfDNA concentration tend to associate with large tumor size and malignant tumors compared to healthy subjects such as Tagawa, 2019 and Kim, 2021. These studies claimed that cfDNA concentration is a good screening tool for the detection of distant metastasis and possible utility when used in combination with existing diagnostic tools. However, the cfDNA concentrations reported in Tagawa and Kim are much higher than the average concentrations observed in other studies that involved larger datasets. Thus, the cfDNA concentration measurement was likely over-estimated and could be contributed by cfDNA and genomic DNA found in cells. As a result, the disclosure in Tagawa is not enabling because they were not successfully measuring cfDNA concentration to make their conclusions. In addition, a small sample size (N<100) with limited representation of breeds and other demographics was used for each analysis to detect differences between the different groups, and might be affected by multiple confounding factors. In contrast, the results presented in this application were generated from a much larger dataset (over thousands of dogs representing diverse demographics), with quantification method specifically developed to target the physical characteristics of cfDNA (for example, taking into consideration the fragment size profiles) unique to cfDNA, and therefore is able to more accurately reflect the association between cfDNA concentrations and the probability of cancer.

Additional studies demonstrated that determination of plasma nucleosome concentration by Nu.Q assay, an ELISA-based test for detecting nucleosome concentration in cfDNA, was a good indicator of cancer progression mainly for systemic cancers (higher metastatic rate). Wilson-Robles, 2022. The Nu.Q assay was only able to correctly detect only half of the cancers for which it was tested, and many additional assays were required to improve the sensitivity of the liquid biopsy techniques in both human and veterinary subjects.

By having a large number of subjects with different types of cancer, and using the screening analyses described herein, the methods disclosed herein can have advantages of having significantly sensitive and optimized cfDNA concentration thresholds for correctly detecting or characterizing cancer or a tumor in a subject.

Embodiments relate to methods for screening subjects for their likelihood to have a cancer or a tumor. In some embodiments, a cancer or a tumor is screened for by isolating a circulating cell free DNA (cfDNA) from a biological sample from a subject, such as a canine, determining the concentration of the cfDNA in the sample, creating a model or summary statistic of the concentration of the cfDNA, comparing the model of the concentration of the cfDNA of the test subject to a second model derived from at least one healthy subject, and determining the presence or absence of the cancer or tumor based upon the comparisons of the two models. Determining the concentration of the cfDNA can be performed through any method recognized by one skilled in the arts, such as quantitative polymerase chain reaction (qPCR). Other non-limiting examples include methods using digital PCR, electrophoresis, or fluorescent assay methods.

In some embodiments, a cancer or a tumor is screened for by the comparison of models. In some embodiments, these models are mixture models. These models are derived from the concentration of the cfDNA. As disclosed herein, screening methods can be performed for the subject suspected of having a cancer or a tumor, as well as a model for one or more healthy subjects, specifically in a subject with difficult-to-diagnose cancers such as lymphoma, osteosarcoma, hemangiosarcoma, histiocytic sarcoma, leukemia, pulmonary malignancy, and cancers of the urinary bladder/urethra. These models can be also specific for sex, size, and age of the subjects. Non-limiting examples of detectable differences include the sensitivity, specificity, and distribution between two models.

A variety of ways exist for determining the concentration of the cfDNA within a subject. In one embodiment, a blood sample is taken from a subject. Circulating cell free DNA (cfDNA) from the blood is obtained. The cfDNA is isolated from the blood cells in the sample. In some embodiments, cfDNA is measured directly from plasma, without isolation or extraction.

Methods provided herein improve the detection, diagnosis, staging, screening, treatment, and management of cancer in subjects, including in canine subjects. In some embodiments, the methods include measuring the concentration of cfDNA directly from a biological sample.

Biological Samples: Some embodiments of the embodiments provided herein measure cfDNA present in a biological sample. Biological samples as used herein include, for example, cell culture media, and tissues and fluids obtained from a subject. A sample obtained from a subject can include any tissue or fluid from the subject that may contain cfDNA. In some embodiments, the biological sample is whole blood, plasma, serum, lymph, vitreous humor, cochlear fluid, tears, peripheral blood, sera, ascites, urine, cerebrospinal fluid (CSF), sputum, saliva, bone marrow, synovial fluid, aqueous humor, amniotic fluid, cerumen, breast milk, bronchoalveolar lavage fluid, semen (including prostatic fluid), Cowper's fluid or pre-ejaculatory fluid, female ejaculate, sweat, fecal matter, hair, cyst fluid, pleural and peritoneal fluid, pericardial fluid, chyme, chyle, bile, interstitial fluid, menses, pus, sebum, vomit, vaginal secretions, mucosal secretion, stool water, pancreatic juice, lavage fluids from sinus cavities, bronchopulmonary aspirates, or other lavage fluids. A biological sample may also include the blastocyl cavity, umbilical cord blood, or maternal circulation, which may be of fetal or maternal origin. The biological sample may also be a tissue sample or biopsy, from which cfDNA may be obtained.

As used herein, “detecting” with respect to measuring a cancer or tumor includes the use of an instrument used to observe and record a signal corresponding to a level or measurement of cancer, or materials required to generate such a signal. In various embodiments, the detecting includes any suitable method, including amplification, sequencing, arrays, fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

It should be realized that the analysis described herein may be part of a larger diagnostic suite used to determine a subject's overall health. For example, the analysis of threshold to determine the concentration of cfDNA in a subject may be used simultaneously or sequentially with other methods for detection, diagnosis, staging, screening, monitoring, treatment, and management of cancer including additional genetic variance analysis. These procedures may be useful to detect a variety of cancers, including lymphoma, leukemia, squamous cell carcinoma, feline mammary cancer, mast cell tumors, bladder cancer, osteosarcoma, hemangiosarcoma, melanoma or a variety of other cancers afflicting subjects.

In some embodiments, the methods include obtaining or having obtained a biological sample from a subject that is suspected of having cancer. In some embodiments, the sample is a liquid biopsy sample, such as a blood sample. In some embodiments, the sample includes cfDNA. In some embodiments, the sample is provided in an amount of less than 10 mL, such as less than 10 mL, 9 mL, 8 mL, 7 mL, 6, mL, 5 mL, 4 mL, 3 mL 2 mL, 1 mL, 500 μL, 250 μL, 100 μL, 50 μL, 25 μL, or 10 μL, or an amount within or less than a range defined by any two of the aforementioned values. In some embodiments, the method includes extracting the cfDNA from the plasma sample. In some embodiments, the method includes determining the concentration of the cfDNA from the plasma sample. Determining the concentration of the cfDNA may be accomplished using techniques, including, for example quantitative PCR (qPCR), digital PCR, fluorescent assay, an automated electrophoresis solution or commercially available kits for cfDNA quantification. In some embodiments, the methods can be used to predict if the subject has cancer or not based on the cfDNA concentration.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there is a plurality of definitions for a term herein, those in this section prevail unless stated otherwise.

As used herein, “a” or “an” can mean one or more than one.

As used herein, the term “about” or “approximately” has its usual meaning as understood by those skilled in the art and thus indicates that a value includes the inherent variation of error for the method being employed to determine a value, or the variation that exists among multiple determinations, and generally refers to a range of numerical values (e.g., +/−5% to 10% of the recited value) that one of ordinary skill in the art would consider equivalent to the recited value (e.g., having the same function or result).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements.

The terms “function” and “functional” as used herein have their plain and ordinary meaning as understood in light of the specification, and refer to a biological, enzymatic, or therapeutic function.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 81, 82, 83, 84, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, the term “circulating” has its plain and ordinary meaning as understood in light of the specification and refers to a substance and/or entity that has been (1) moving or causing to move continuously or freely through a closed system or area, and/or (2) pass or cause to pass from place to place or person to person.

As used herein, the term “extracting” has its plain and ordinary meaning as understood in light of the specification and refers to a substance and/or entity that has been (1) removing or taking out by effort or force, (2) obtaining a substance or resource from something by a special method. Extracting the circulating cell free DNA from a plasma sample indicates removing the cfDNA from the blood plasma after centrifugation using different extraction kits such as CNA kit, RSC kit and ME kit.

As used herein, the term “determining” has its usual meaning as understood by those skilled in the art and thus indicates (1) causing something to occur or be done in a particular way, (2) to find out or come to a decision about by investigation, reasoning, or calculation.

As used herein, the term “generating” has its usual meaning as understood by those skilled in the art and thus indicates (1) causing something to arise or come about, (2) to produce a set or sequence by performing specified mathematical or logical operations on an initial set.

As used herein, the term “concentration” has its ordinary meaning as understood in light of the specification and refers to (1) the amount of a dissolved substance that is in a unit volume, (2) the ratio of solute in a solution to either solvent or total solution. Concentration is usually expressed in terms of mass per unit volume. The unit examples of concentration: g/cm3, kg/l, M, m, N, kg/L. A concentrated solution refers to a chemical solution with large amount of solute in it. A diluted solution reference to a chemical solution with low amount of solute dissolved in it. The concentration of the DNA (DNA) can be measured in few different ways such as ultraviolet (UV) absorbance, fluorescence, and diphenylamine reaction.

As used herein, “nucleic acid,” “nucleic acid molecule,” or “nucleotide” refers to polynucleotides or oligonucleotides such as deoxyribonucleic acid (DNA) or ribonucleic acid (RNA), oligonucleotides, fragments generated by the polymerase chain reaction (PCR), and fragments generated by any of ligation, scission, endonuclease action, exonuclease action, and by synthetic generation. Nucleic acid molecules can be composed of monomers that are naturally occurring nucleotides (such as DNA and RNA), or analogs of naturally occurring nucleotides (e.g., enantiomeric forms of naturally occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded.

The terms “peptide,” “polypeptide,” and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95,100,150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The terms “cancer” and “cancerous” have their ordinary meaning as understood in light of the specification and refer to or describe the physiological condition in animals that is typically characterized by unregulated cell growth. A “tumor” comprises one or more cancerous cells. In some embodiments, the tumor is a solid tumor. There are several main types of cancer. Carcinoma is a cancer that originates from epithelial cells, for example skin cells or lining of intestinal tract. Sarcoma is a cancer that originates from mesenchymal cells, for example bone, cartilage, fat, muscle, blood vessels, or other connective or supportive tissue. Leukemia is a cancer that originates in hematopoietic cells, such as the bone marrow, and causes large numbers of abnormal blood cells to be produced and enter the blood. Lymphoma and multiple myeloma are cancers that originate in the lymphoid cells of lymph nodes. Central nervous system cancers are cancers that originate in the central nervous system and spinal cord. In some embodiments, cancers include those that are specific to a companion animal, such as to a dog, for example. Examples of cancers may include lymphoma, hemangiosarcoma, soft tissue sarcoma, mast cell tumor, osteosarcoma, mammary gland carcinoma, anal sac adenocarcinoma, and malignant melanoma. In some embodiments, the test is pan-cancer, an analysis across diverse tumor types.

In some embodiments, the methods provided herein assign subjects to a probability tier, referred to herein as a cancer probability index. As used herein, the term “cancer probability index” refers to a probability of cancer upon performance of the methods described herein, and can include low, moderate, or high probabilities, or probabilities within a range of these terms. For example, a low probability may include a likelihood of having a reduced likelihood of cancer compared to cancer prevalence. A reduced likelihood of having cancer can be reduced by a factor of 2 or 3. A moderate probability may include a likelihood of having cancer increased by a slight amount, such as by 10%, 20%, 30%, 40%, 50%, or 60%, or an amount within a range defined by any two of the aforementioned values, as compared to cancer prevalence. A high probability may include a likelihood of having cancer increased by a significant amount, such as by more than 70%, 80%, 90%, 100%, 150%, 200%, 300%, 400%, 500%, 600%, 700%, or greater, or an amount within a range defined by any two of the aforementioned values, as compared to cancer prevalence.

As used herein, the term “amplification” has its ordinary meaning as understood in light of the specification and refers to any methods known in the art for copying a target nucleic acid, thereby increasing the number of copies of a selected nucleic acid sequence. Amplification may be exponential or linear. A target nucleic acid may be either DNA or RNA. Typically, the sequences amplified in this manner form an “amplicon.” Amplification may be accomplished with various methods including, but not limited to, the polymerase chain reaction (“PCR”), transcription-based amplification, isothermal amplification, rolling circle amplification, etc. Amplification may be performed with relatively similar amount of each primer of a primer pair to generate a double stranded amplicon. However, asymmetric PCR may be used to amplify predominantly or exclusively a single stranded product as is well known in the art (e.g., Poddar et al. Molec. And Cell. Probes 14:25-32 (2000)). This can be achieved using each pair of primers by reducing the concentration of one primer significantly relative to the other primer of the pair (e.g., 100-fold difference). Amplification by asymmetric PCR is generally linear. A skilled artisan will understand that different amplification methods may be used together.

The terms “individual,” “subject,” “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs.

As used herein, the term “liquid biopsy” has its ordinary meaning as understood in light of the specification and refers to the collection of a sample and the testing the sample, wherein the sample is non-solid biological tissue such as blood.

As used herein, the term “cfDNA” has its ordinary meaning as understood by those of skill in the art, and refers to circulating cell free DNA, which includes DNA fragments released to the blood plasma. cfDNA can include circulating tumor deoxyribonucleic acid (ctDNA).

As used herein, the term “plasma” has its ordinary meaning as understood in light of the specification and refers to the clear, yellowish, liquid portion of the blood which is made of 90-92% water. Fifty-five percent of the blood is made of plasma and the other 45% are red blood cells, white blood cells and platelets. Plasma serves as a transport medium for delivering nutrients and proteins to the cells of the various organs of the body and for transporting waste products derived from cellular metabolism to the kidneys, liver, and lungs for excretion. It is also a transport system for blood cells. Plasma helps to distribute heat throughout the body and to maintain homeostasis, or biological stability, including acid-base balance in the blood and body. Plasma also contains 6-8% proteins. One critical group is the coagulation proteins and their inhibitors, synthesized primarily in the liver.

As used herein, the term “sensitivity” has its ordinary meaning as understood by those of skill in the art, and refers to the true positive rate, which is the probability of a positive test result, conditioned on the subject truly being positive.

As used herein, the term “specificity” has its ordinary meaning as understood in light of the specification, and refers to the true negative rate, which is the probability of a negative test result, conditioned on the individual truly being negative.

As used herein, the term “Screening Concept 1” refers to a population where only the cfDNA concentration is measured.

As used herein, the term “Screening Concept 2” refers to a population in which subjects with moderately high cfDNA concentrations are further evaluated using the OncoK9 genomic test. OncoK9 is a cell-free DNA based noninvasive genomic cancer screening test for canine cancer detection, powered by next-generation sequencing (NGS) and using bioinformatic analysis. OncoK9 is capable of detecting various cancers, including, for example, lymphoma, hemangiosarcoma, soft tissue sarcoma, mast cell tumor, osteosarcoma, mammary gland carcinoma, anal sac adenocarcinoma, and/or malignant melanoma. In some embodiments, the methods include performing a genomic cancer screening assay on a subject classified as moderate. In some embodiments, the genomic cancer screening assay is an OncoK9 assay.

As used herein, the term “distribution” has its ordinary meaning as understood in light of the specification, and refers to the position, arrangement, or frequency of occurrence (as of the members of a group) over an area or throughout a space or unit of time. In statistics, distribution is a mathematical function that describes the relationship of observations of different heights. A distribution is simply a collection of data, or scores, on a variable. It describes the probability that a system will take on a specific value or set of values. The highest point on the curve indicates the most common or modal value, which in most cases will be close to the average (mean) for the population. As used herein, the term “cfDNA distribution” refers to the frequency of occurrence, and may include distribution of cfDNA across different cancers and/or across different subjects.

The above description discloses several methods and materials. This disclosure is susceptible to modifications in the methods and materials, as well as alterations in the fabrication methods and equipment. Such modifications will become apparent to those skilled in the art from a consideration of the disclosure disclosed herein. Consequently, it is not intended that embodiments described herein be limited to the specific embodiments disclosed herein, but that it covers all modifications and alternatives coming within the true scope and spirit of the disclosure.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

In another embodiment of the methods described herein, any of the methods described herein can be used alone, or any of the methods described herein can be used in combination with any other method or methods described herein.

### EXAMPLES

Embodiments of the present invention are further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims. The disclosure of each reference set forth herein is incorporated herein by reference in its entirety, and for the disclosure referenced herein.

### Example 1

### cfDNA Extraction from Subjects

Embodiments of cfDNA isolation described herein was performed using a series of extractions. Blood samples were collected from canine subjects into anti-coagulant blood collection tubes (BCTs) containing circulating cell free DNA stabilizing components. Non-limiting examples of viable collection tubes include the Roche Cell-Free DNA collection tubes, as well as Streck, Biomatrica, MagMax, or Norgen collection tubes. BCTs were then centrifuged to separate the plasma fraction and red and white blood cells. The cell free plasma layer was removed from the BCT and either stored or taken directly into circulating cell free DNA (cfDNA) extraction.

cfDNA was extracted from 2-8 mL of plasma using a commercially available magnetic bead-based extraction kit (MagMax Cell-Free DNA Isolation Kit). Other comparable extraction methods/kits could potentially be used for this process, including column-based solid phase methods, and well as precipitation-based methods. cfDNA was eluted and the concentration of cfDNA was quantified by automated electrophoresis (TapeStation by Agilent, for example). The choice of the type of assay performed in the electrophoresis platform and the specific parameters used for the quantification were optimized to target the specific fragment size profiles of cell-free DNA. Other ways of quantifying the concentration of cfDNA was by quantitative PCR (qPCR) or digital PCR, and may be performed by directly measuring from the plasma sample without DNA extraction.

Whole genome libraries were prepared from the cfDNA by contacting the cfDNA sample with random primers configured to amplify whole genomes for sequencing. However, it will be understood to those skilled in the art that any method suitable for sequence amplification could be utilized, such as next generation sequencing, for example. In one embodiment, library preparation may include incorporation of unique molecular identifiers and unique sample specific barcodes to allow for multiplexing of samples from different subjects.

### Example 2

### Analysis of the Demographics of Cancer and Cancer-Free Subjects

Embodiments of analysis of the subject's cfDNA was performed through a comparison of the sex, age, and weight of canines with all types of cancer, and the canines with difficult-to-diagnose (D2D) cancer compared to those in cancer-free canine subjects. The dataset consisted of 754 cancer-diagnosed subjects and 1204 cancer-free subjects. Among the 754 cancer-diagnosed subjects, 324 were diagnosed with a relevant D2D cancer. Table 1 shows the demographics for cancer-diagnosed (all cancers and D2D cancers separately) and cancer-free subjects.

Samples were split into a 2:1 ratio for training and testing by cancer status. That is, two thirds of cancer-diagnosed samples were randomly assigned to training and the remainder for testing, and likewise for cancer-free samples. After this split, the training set was comprised of 502 cancer-diagnosed samples (229 of which were D2D cancers) and 802 cancer-free samples, while the testing set was comprised of 252 cancer-diagnosed samples (95 of which were D2D cancers) and 402 cancer-free samples.

TABLE 1
Demographics for cancer-diagnosed (all cancers and
D2D cancers separately) and cancer-free subjects

| | Cancer- | Cancer-diagnosed | |
|---|---|---|---|
| | diagnosed | (D2D | |
| | (all cancers) | cancers) | Cancer-free |
| Total N | 754 | 324 | 1204 |
| N Male Subjects (%) | 407 (54.0%) | 187 (57.7%) | 613 (51.0%) |
| Mean \| Median Age | 9.7 \| 10.0 | 9.1 \| 9.2 | 6.4 \| 6.1 |
| (min-max; years) | (1.3-17.0) | (1.5-15.9) | (1.0-17.3) |
| Mean \| Median Weight | 27.2 \| 27.8 | 29.6 \| 29.3 | 25.9 \| 26.3 |
| (min-max; kg) | (1.8-81.7) | (5.0-81.7) | (3.0-106.6) |

### Example 3

### Analysis to Optimize the Sensitivity and Specificity Across Different Cohorts of Cancer

The following example demonstrates performing data analysis on cfDNA to determine whether different cancer sets would produce more robust data compared to the cancer as a whole.

The cancers were divided into different groups—all cancers, Nu.Q™ cancer (included in Nu.Q™ marketing materials: lymphoma, osteosarcoma, hemangiosarcoma, soft tissue sarcoma, histiocytic sarcoma, mast cell tumor, malignant melanoma), Nu.Q+™ cancers (included in Nu.Q™ marketing materials plus additional cancer types where commercial OncoK9 assays performs well per the international clinical cancer detection in dogs (CANDiD) study: lymphoma, osteosarcoma, hemangiosarcoma, soft tissue sarcoma, histiocytic sarcoma, mast cell tumor, malignant melanoma, leukemia, mammary gland carcinoma, lung), and D2D cancers (include lymphoma, osteosarcoma, hemangiosarcoma, histiocytic sarcoma, leukemia, pulmonary malignancy, and cancers of the urinary bladder/urethra).

The lower and upper threshold combinations of the cfDNA concentration was manually selected to optimize the overall sensitivity and specificity of the data analysis.

Sensitivity and specificity were calculated using the following formula:

Sensitivity (Concept 1)={#Cancer-diagnosed subjects with High Probability}/{#Cancer-diagnosed subjects with High or Low Probability}

Specificity (Concept 1)={#Cancer-free subjects with Low Probability}/{#Cancer-free subjects with High or Low Probability}

During the analysis of the Screening Concept 1, the calculations exclude the fraction of subjects with moderate probability results, while during the analysis of the Screening Concept 2 these samples are included since all Moderate Probability results are automatically tested by OncoK9/OKR prior to reporting.

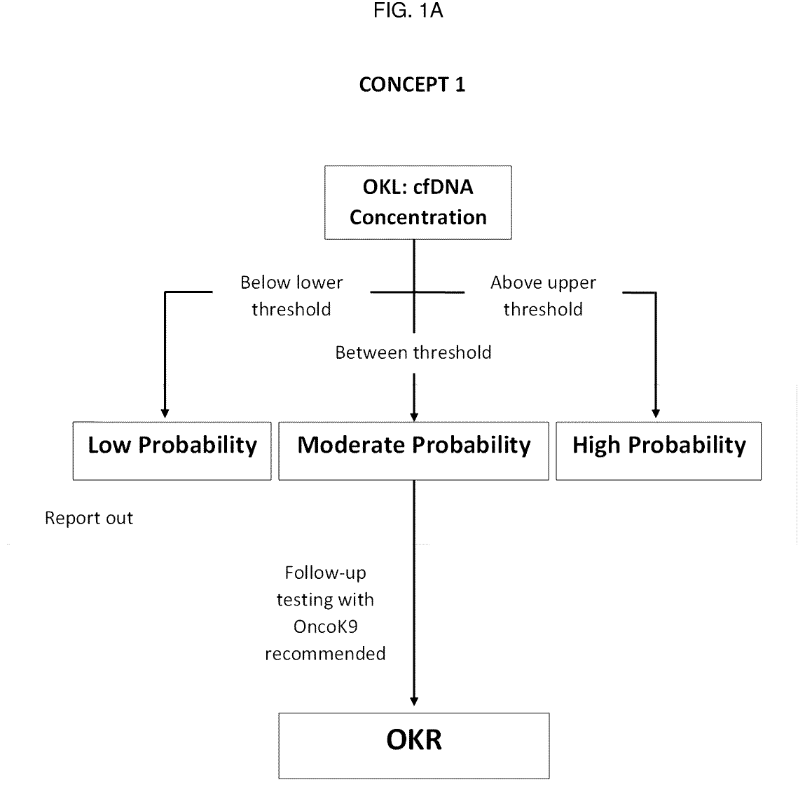

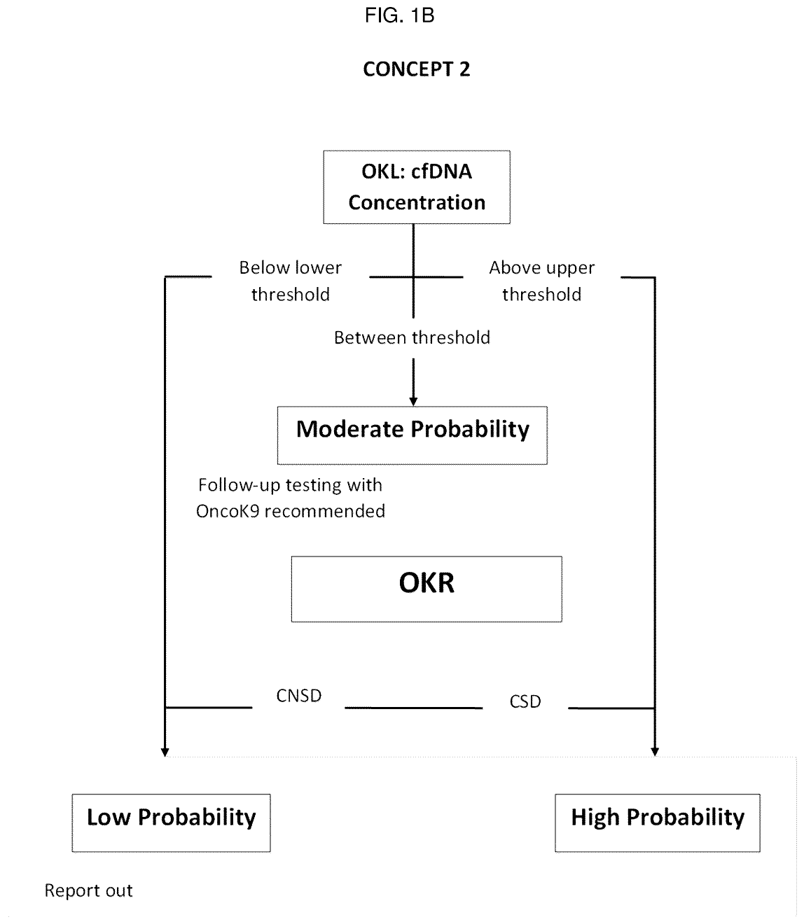

In the Screening Concept 1, these calculations exclude the fraction of subjects with Moderate Probability results, while for Screening Concept 2 these samples are included since all Moderate Probability results are automatically tested by OncoK9 prior to reporting shown in FIGS. 1A and 1B. In the figures, “OKR” refers to OncoK9 genomic test; “CSND” refers to cancer signal not detected; “CSD” refers to cancer signal detected; and “OKL” refers to Concepts 1 or 2.

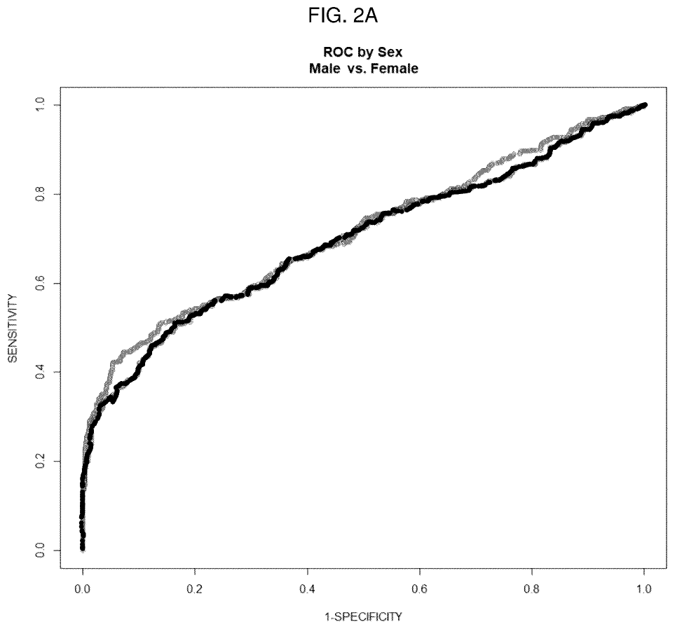

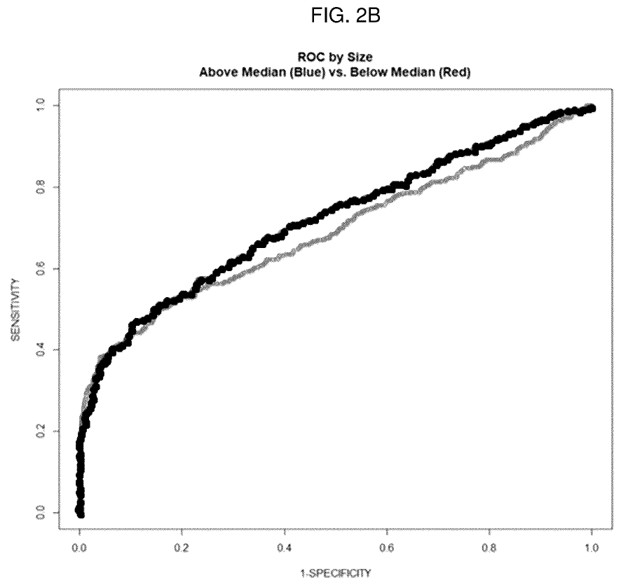

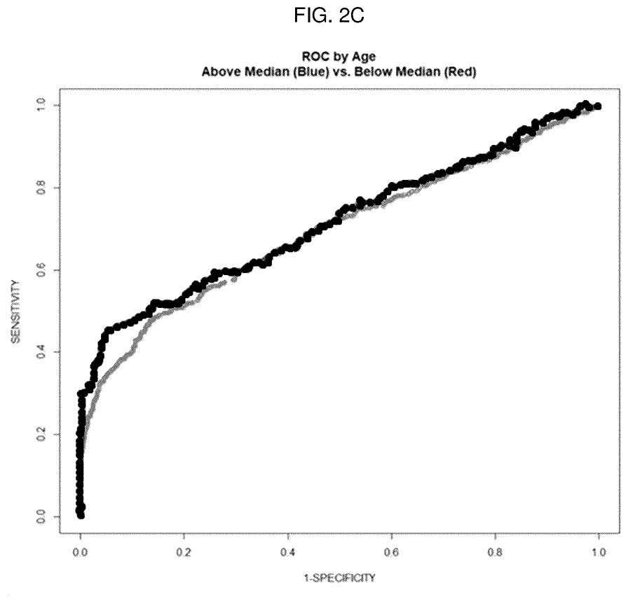

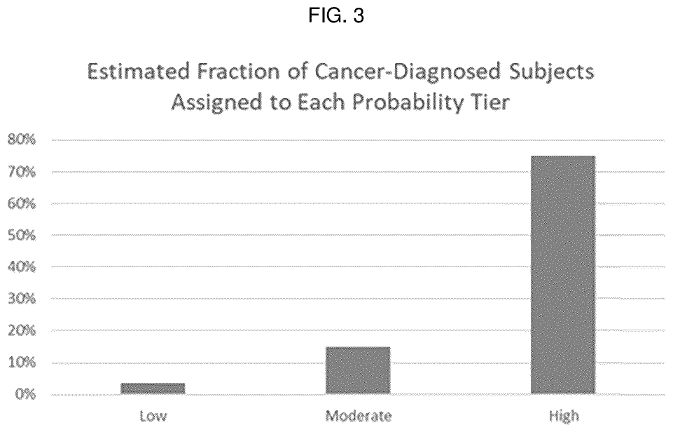

A receiver operator characteristic (ROC) curve was generated for the different demographic variables (sex, weight, age). There was no significant difference in performance was observed in the ROC curve as a function of the demographic variables as shown in FIGS. 2A-2C. However, the optimal points on the curves does not correspond to the same cfDNA concentration threshold. Instead, the sex-specific thresholds performed better than the single thresholds.

A top threshold may be selected to optimize sensitivity and specificity on the Nu.Q+™ cancer set while minimizing the number of samples binned into the moderate probability group. The final upper and lower thresholds for the different sex are listed in Table 2 with grid search results.

TABLE 2
Final cfDNA concentration thresholds based on reported sex.

| | Lower Threshold (ng/mL) | Upper Threshold (ng/mL) |
|---|---|---|
| Male | 0.9 | 1.9 |
| Female | 0.8 | 1.7 |

### Example 4

### Analysis of Sensitivity Across Different Groups of Cancer

The following example demonstrates a summary of the comparison of the Concept 1 and 2 in different groups of cancer by measuring the sensitivity and the specificity and the moderate Cancer Probability Index results.

The tables below demonstrate the overall performance of the Screening Concepts 1 and 2 for both training (Table 3) and testing (Table 4) sets when applied to D2D cancers.

TABLE 3
Performance for Screening Concepts 1 and 2 in training set

| | Sensitivity (D2D cancer set) | Specificity | Moderate Probability Results (%) |
|---|---|---|---|
| Concept 1 | 63.9% | 97.7% | 13.8% |
| Concept 2 | 65.5% | 97.3% | 13.8% |

TABLE 4
Performance for Screening Concepts 1 and 2 in testing set.

| | | | Moderate Probability Results |
|---|---|---|---|
| | Sensitivity (D2D cancer set) | Specificity | (%) |
| Concept 1 | 66.7% (50/75) | 97.7% | 13.7% |
| | | (342/350) | |
| Concept 2 | 69.5% (66/95) | 97.5% | 13.7% |
| | rounds to 69% | (392/402) | |

### Example 5

### Comparison of Sensitivity Between Different Groups of Cancer

The following example demonstrates a summary of the comparison of the sensitivity of different cancers between the different groups of cancer.

The sensitivity across both training and testing sets for each cancer type in the D2D cancer set was carried out and is demonstrated in Table 5. The subjects with multiple diagnoses were included in Table 5 if at least one of their diagnoses was the given cancer. A single subject maybe selected to be included in both cancer's performance estimates if diagnosed with two D2D cancers.

TABLE 5
Training + Testing set performance for each cancer type in the D2D set.

| Cancer | Concept 1 Sensitivity | | Concept 2 Sensitivity | |
|---|---|---|---|---|
| Lymphoma, Intermediate to Large Cell | 83.0% | (112/135) | 84.0% | (126/150) |
| Hemangiosarcoma | 62.5% | (15/24) | 64.7% | (22/34) |
| Leukemia (ALL or CLL) | 57.1% | (12/21) | 63.0% | (17/27) |
| Bone, Osteosarcoma | 50.0% | (26/52) | 58.0% | (40/69) |
| Pulmonary Malignancy | 36.4% | (4/11) | 43.8% | (7/16) |
| Histiocytic Sarcoma | 30.8% | (4/13) | 29.4% | (5/17) |
| Cancers of the Urinary Bladder/Urethra | 21.1% | (4/19) | 20.0% | (4/20) |

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through second aspects is applicable to all aspects and embodiments identified herein. Moreover, any of the features of an embodiment of the first through third aspects is independently combinable, partly or wholly with other embodiments described herein in any way, e.g., one, two, or three or more embodiments may be combinable in whole or in part. Further, any of the features of an embodiment of the first through third aspects may be made optional to other aspects or embodiments.

What is claimed is:

1. A method of detecting a cancer or tumor in a subject, the method comprising:
isolating circulating cell free DNA (cfDNA) from a sample from the subject;
extracting the cfDNA from the sample;
determining a concentration of the cfDNA from the sample;
generating a first experimental model from one or more cfDNA concentration distributions of subjects with cancer;
generating a second experimental model from one or more cfDNA concentration distributions of subjects without cancer; and
determining the presence of the cancer or tumor based upon the comparison of the first and second models to the concentration of the subjects cfDNA.

2. The method of claim 1, wherein the sample is blood, plasma, urine, saliva, effusion, or cerebral spinal fluid.

3. The method of claim 1, wherein the determination of the concentration of the cfDNA is done using an electrophoresis solution.

4. The method of claim 1, wherein the determination of the concentration of the cfDNA is done using quantitative PCR (qPCR), digital PCR, or fluorometric assays.

5. The method of claim 1, wherein one or more optimized threshold is obtained from the first and second experimental model.

6. The method of claim 5, wherein the one or more optimized threshold classifies the concentration of the cfDNA into a low, moderate, and high category.

7. The method of claim 6, further comprising performing a genomic cancer screening assay on a subject classified as moderate.

8. The method of claim 5, wherein the one or more optimized threshold is different for a given demographic variable.

9. The method of claim 8, wherein the demographic variable is subject gender, subject size, subject age, subject breed, and/or spay or neutered subjects.

10. The method of claim 5, wherein the one or more optimized threshold is a single threshold, and wherein the concentration of the cfDNA above the threshold is a prediction of cancer.

11. The method of claim 1, wherein the subject is a mammal.

12. The method of claim 11, wherein the subject is a canine, feline, equine, or human.

13. The method of claim 1, wherein the cancer is lymphoma, hemangiosarcoma, soft tissue sarcoma, mast cell tumor, osteosarcoma, mammary gland carcinoma, anal sac adenocarcinoma, and/or malignant melanoma.

14. A method of detecting a cancer or tumor in a subject, the method comprising:
obtaining a biological sample comprising circulating cell free DNA (cfDNA) from the subject;
determining a concentration of the cfDNA directly from the sample;
generating a first experimental model from one or more cfDNA concentration distributions of subjects with cancer;
generating a second experimental model from one or more cfDNA concentration distributions of subjects without cancer; and
determining the presence of the cancer or tumor based upon the comparison of the first and second models to the concentration of the subjects cfDNA.

15. The method of claim 14, wherein the sample is blood, plasma, urine, saliva, effusion, or cerebral spinal fluid.

16. The method of claim 14, wherein the determination of the concentration of the cfDNA is done using an electrophoresis solution.

17. The method of claim 14, wherein the determination of the concentration of the cfDNA is done using quantitative PCR (qPCR), digital PCR, or fluorometric assays.

18. The method of claim 14, wherein one or more optimized threshold is obtained from the first and second experimental model.

19. The method of claim 18, wherein the one or more optimized threshold classifies the concentration of the cfDNA into a low, moderate, and high category.

20. The method of claim 19, further comprising performing a genomic cancer screening assay on a subject classified as moderate.

21. The method of claim 18, wherein the one or more optimized threshold is different for a given demographic variable.

22. The method of claim 21, wherein the demographic variable is subject gender, subject size, subject age, subject breed, and/or spay or neutered subjects.

23. The method of claim 18, wherein the one or more optimized threshold is a single threshold, and wherein the concentration of the cfDNA above the threshold is a prediction of cancer.

24. The method of claim 14, wherein the subject is a mammal.

25. The method of claim 24, wherein the subject is a canine, feline, equine, or human.

26. The method of claim 14, wherein the cancer is lymphoma, hemangiosarcoma, soft tissue sarcoma, mast cell tumor, osteosarcoma, mammary gland carcinoma, anal sac adenocarcinoma, and/or malignant melanoma.
